(19) United States
(12) Patent Application Publication
(10) Pub. No.: US 2017/0087266 A1
(43) Pub. Date: Mar. 30, 2017

(54) AIR FRESHENER DISPENSERS, CARTRIDGES THEREFOR, ASSEMBLIES, AND METHODS

(71) Applicant: Georgia-Pacific Consumer Products LP, Atlanta, GA (US)

(72) Inventors: Gordon Keith Graham, Omro, WI (US); Daniel M. D'Amico, South Salem, NY (US); Jacob Edward Malesky, Neenah, WI (US); Eric Paul Peterson, Hortonville, WI (US); Adria Rose Griffin, Lombard, IL (US); Ryan Allen Andersen, Appleton, WI (US)

(21) Appl. No.: 15/279,551

(22) Filed: Sep. 29, 2016

Related U.S. Application Data
(60) Provisional application No. 62/234,207, filed on Sep. 29, 2015.

(51) Int. Cl. A61L 9/012

(52) U.S. Cl. CPC A61L 9/012; A61L 2209/15

(57) ABSTRACT

Air freshener cartridges and assemblies are provided herein. An air freshener cartridge includes a body formed of a polymer material impregnated with an air freshening substance for release, the body having a plurality of walls defining a plurality of axial airflow channels therebetween, and a base member including a support frame in contact with a surface of the body and for loading the cartridge into the air freshener dispenser and unloading the cartridge from the air freshener dispenser. An air freshener assembly includes an air freshener cartridge and an air freshener dispenser having a housing with a support member defining a cavity for removably receiving the cartridge, and a fan within the housing operable to induce an airflow through the axial airflow channels of the body, to direct the airflow to an area outside of the air freshener dispenser.

### CROSS-REFERENCE TO RELATED APPLICATION

This application claims priority to U.S. Provisional Application No. 62/234,207, filed Sep. 29, 2015, which is incorporated by reference herein.

### TECHNICAL FIELD

The present disclosure relates generally to air freshener dispensers and cartridges therefor, and more particularly relates to air freshener dispensers and cartridges therefor having a base supporting the air freshener material.

### BACKGROUND

There are numerous problems and frustrations relating to air care and malodor that exist in public washrooms today. To combat malodor, public washroom owners currently rely on a variety of options; however, the current options are expensive, ineffective, or difficult to manage. For example, wall-mounted aerosol systems are the most commonly used option to address malodor. Aerosols often are effective at dispersing fragrance molecules or droplets into a large space quickly, but are messy because a liquid is sprayed into the air. The refill (typically, a canister) is difficult to dispose of, and transportation, packaging, and storage of canisters is a challenge because of their combustion potential. Wall-mounted diffusion (both static and active) systems are new to the public washroom air care space. They are somewhat effective at treating malodor, but experience certain performance issues and refilling them is costly and difficult.

In particular, wall-mounted diffusion systems are difficult to access and refill because they are typical located out of reach (e.g., high on a wall) to limit pilfering and to improve efficacy throughout the washroom. Additionally, passive diffusion systems are limited in their ability to diffuse air freshener to a large area, such as high-traffic washroom settings. Such systems typically result in regions of high fragrance near the unit while leaving areas farther from the unit untreated. Traditional active diffusion systems commonly consist of a fan forcing air over a refill, such as a cup of air freshening liquid or other material. Air flow around and over these refills causes undue turbulence, limiting the effective range of the air freshening unit. Accordingly, there is a need for improved devices and methods to freshen the air and to reduce malodor in public washrooms. Therefore, it is to these ends, as well as other benefits, that the presently disclosed dispensers and cartridges are directed.

### SUMMARY

In one aspect, air freshener cartridges for use in an air freshener dispenser are provided. In one embodiment, the air freshener cartridge includes a body formed of a polymer material impregnated with an air freshening substance for release, the body having a plurality of radial spokes and a plurality of parallel walls intersecting the spokes and defining a plurality of axial airflow channels therebetween. The air freshener cartridge further includes a base member including a support frame in contact with a surface of the body that is perpendicular to an axis of the body, the support frame defining one or more airflow apertures, such that at least a portion of the axial airflow channels of the body align with the airflow apertures of the base member. The base member includes a gripping surface for a user to load the cartridge into the air freshener dispenser and to unload the cartridge from the air freshener dispenser.

In another embodiment, an air freshener cartridge for use in an air freshener dispenser includes a body formed of ethylene-vinyl acetate impregnated with an air freshening substance for release, the body having a plurality of walls defining a plurality of axial airflow channels therebetween, and a base member including a support frame in contact with a surface of the body and for loading the cartridge into the air freshener dispenser and unloading the cartridge from the air freshener dispenser, the base member formed of a composition free of the air freshening substance, wherein the body has a unitless surface area to volume ratio of at least about 4.

In another aspect, air freshener dispenser assemblies are provided. In one embodiment, an air freshener dispenser assembly includes (i) an air freshener cartridge that includes a body formed of a polymer material impregnated with an air freshening substance for release, the body having a plurality of walls defining a plurality of axial airflow channels therebetween, and a base member including a support frame in contact with a surface of the body that is perpendicular to an axis of the body, the support frame defining one or more airflow apertures, such that at least a portion of the axial airflow channels of the body align with the airflow apertures of the base member, and (ii) an air freshener dispenser that includes: a housing having at least one support member defining a cavity for removably receiving the cartridge, a fan within the housing operable to induce an airflow through the aligned axial airflow channels of the body and airflow apertures of the base member, to direct the airflow to an area outside of the air freshener dispenser, such that released air freshening substance is entrained in the airflow directed from the air freshener dispenser, and a motor within the housing for driving the fan.

In another embodiment, an air freshener dispenser assembly includes (i) an air freshener cartridge that includes a body formed of ethylene-vinyl acetate impregnated with an air freshening substance for release, the body having a plurality of walls defining a plurality of axial airflow channels therebetween, and a base member including a support frame in contact with a surface of the body that is perpendicular to an axis of the body and for loading the cartridge into the air freshener dispenser and unloading the cartridge from the air freshener dispenser, and (ii) an air freshener dispenser that includes a housing having at least one support member defining a cavity for removably receiving the cartridge, such that the body of a cartridge loaded in the cavity does not contact the support member, a fan within the housing operable to induce an airflow through the axial airflow channels of the body, and to direct the airflow to an area outside of the air freshener dispenser, such that released air freshening substance is entrained in the airflow directed from the air freshener dispenser, and a motor within the housing for driving the fan.

### BRIEF DESCRIPTION OF THE DRAWINGS

Referring now to the drawings, which are meant to be exemplary and not limiting, and wherein like elements are numbered alike. The detailed description is set forth with reference to the accompanying drawings illustrating examples of the disclosure, in which use of the same reference numerals indicates similar or identical items. Certain embodiments of the present disclosure may include elements, components, and/or configurations other than those illustrated in the drawings, and some of the elements, components, and/or configurations illustrated in the drawings may not be present in certain embodiments.

FIG. 1A is a perspective front view of a closed air freshener dispenser, in accordance with the present disclosure.

FIG. 1B is a perspective bottom view of the closed air freshener dispenser of FIG. 1A.

FIG. 1C is an internal perspective view of the open air freshener dispenser of FIG. 1A.

FIG. 1D is a rear perspective view of the closed air freshener dispenser of FIG. 1A.

FIG. 2 is a partial perspective internal view of an air freshener assembly including an air freshener dispenser and an air freshener cartridge, in accordance with the present disclosure.

FIG. 3A is an exploded side view of an air freshener cartridge, in accordance with the present disclosure.

FIG. 3B is an exploded upper perspective view of the air freshener cartridge of FIG. 3A.

FIG. 4A is a top view of a base member for an air freshener cartridge, in accordance with the present disclosure.

FIG. 4B is a side view of the base member of FIG. 4A.

FIG. 4C is an upper perspective view of the base member of FIG. 4A.

FIG. 5A is a top view of a base member for an air freshener cartridge, in accordance with the present disclosure.

FIG. 5B is a side view of the base member of FIG. 5A.

FIG. 5C is an upper perspective view of the base member of FIG. 5A.

FIG. 6A is a top view of a base member for an air freshener cartridge, in accordance with the present disclosure.

FIG. 6B is a side view of the base member of FIG. 6A.

FIG. 6C is an upper perspective view of the base member of FIG. 6A.

FIG. 7 is an exploded upper perspective view of an air freshener cartridge, in accordance with the present disclosure.

FIG. 8 is an upper perspective view of a base member for an air freshener cartridge, in accordance with the present disclosure.

FIG. 9A is a top plan view of an embodiment (V1) of a cartridge body for an air freshener cartridge that was manufactured and tested in the Example.

FIG. 9B is a top plan view of an embodiment (V2) of a cartridge body for an air freshener cartridge that was manufactured and tested in the Example.

FIG. 9C is a top plan view of an embodiment (V3) of a cartridge body for an air freshener cartridge that was manufactured and tested in the Example.

FIG. 9D is a top plan view of an embodiment (V4) of a cartridge body for an air freshener cartridge that was manufactured and tested in the Example.

FIG. 10 is a graph showing the results of fragrance intensity over time for air freshener cartridges containing Scent 1 tested in the Example.

FIG. 11 is a graph showing the results of fragrance intensity over time for air freshener cartridges containing Scent 2 tested in the Example.

FIG. 12 is a graph showing the results of fragrance intensity over time for air freshener cartridges containing Scent 3 tested in the Example.

FIG. 13 is a graph showing the results of fragrance intensity over time for air freshener cartridges containing Scent 4 tested in the Example.

FIG. 14 is a graph showing the results of fragrance intensity over time for air freshener cartridges containing Scent 5 tested in the Example.

FIG. 15 is a graph showing the results of fragrance intensity over time for air freshener cartridges containing Scent 6 tested in the Example.

### DETAILED DESCRIPTION

Disclosed herein are active air freshener dispensers and cartridges therefor, as well as assemblies of these dispensers and cartridges. These dispenser assemblies and cartridges may have one or more of the following benefits over currently available options: (1) improved effectiveness because the dispenser/freshener releases the air freshener in a controlled and predictable manner; (2) easier refillability because the dispenser and cartridge may include a self-locating feature; (3) easier cartridge handling and refillability because the cartridge includes a base for handling the air freshener; (4) easier maintenance because the dispenser/freshener does not require a liquid refill; and (5) improved cartridge lifespan and fragrance intensity over the lifespan.

Embodiments of the fragrance dispenser systems also solve the problem of combatting malodor without releasing an excessive amount of fragrance, which itself can be perceived by washroom occupants/users as offensive and which can prematurely deplete the cartridge. As described herein, the problem is solved by a combination of the freshener cartridge configuration and dispenser configuration, to achieve a desirable quantity and rate of release of the fragrance. Effective configurations were developed from testing.

Air Freshener Dispensers

Active air freshener dispensers are provided herein. In certain embodiments, the active air freshener dispensers are configured to be mounted to a wall or other surface, within a washroom. As used herein, the terms “air freshener” and “air freshening” refer to substances that treat air by combatting or neutralizing odor, providing a fragrance, or both.

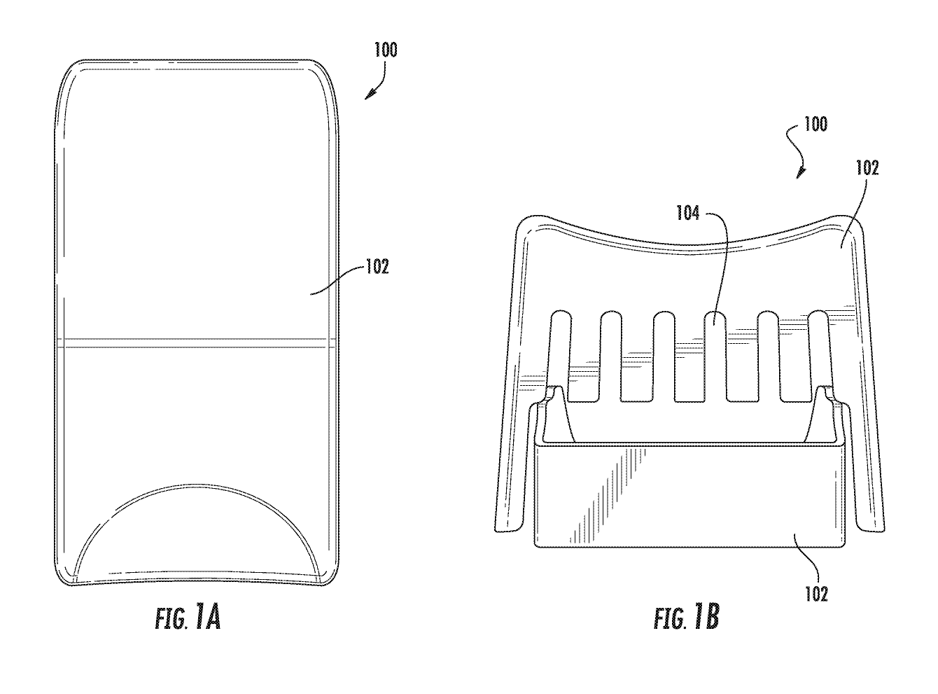

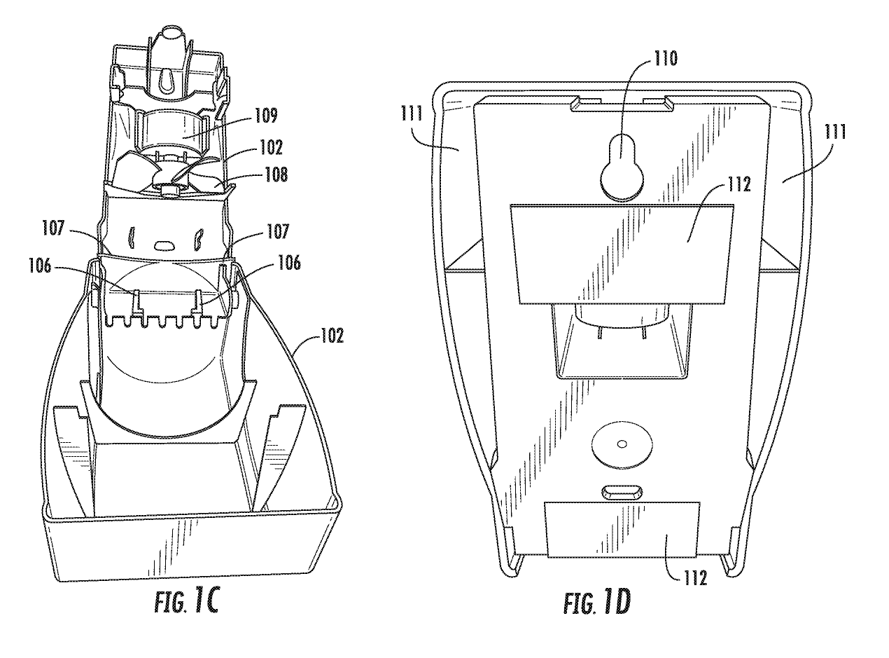

FIGS. 1A-1D show front, bottom, internal, and rear views of one embodiment of an air freshener dispenser 100 in accordance with this disclosure. In certain embodiments, the housing 102 of the active air freshener dispenser is mountable to a wall or other washroom surface, such as a vanity or washroom stall surface. For example, the housing of the air freshener dispenser may be configured for attachment to a surface by adhesives, solder, welding, magnetic connection, hook-and-loop fasteners, mechanical fasteners, such as screws or bolts, and the like. FIG. 1D shows an embodiment of the air freshener dispenser in which the rear wall of the housing includes a screw-hole 110 and strips of double-sided adhesive tape 112, for mounting.

FIG. 1A shows the front of the housing. FIG. 1B shows the bottom, or base, of the housing 102, including an outlet vent 104 through which at least a portion, if not all, of the airflow directed from the air freshener dispenser 100 that contains released air freshening substance from the cartridge, is directed. In certain embodiments, the airflow outlet has an open area of from about 1,000 mm² to about 1,500 mm². In one embodiment, the outlet has an open area of about 1,200 mm². As used herein, the term “about” means plus or minus 10 percent of the numerical value of the number with which it is being used.

FIG. 1C shows the inside of the housing 102 of an air freshener dispenser that has not yet been loaded with an air freshener cartridge. The air freshener dispenser includes a hinge such that the front portion of the housing 102 can be rotated into an open position, to provide access to the interior of the housing. The air freshener dispenser housing 102 includes at least one support member 106 that defines a cavity, or channel, for removably receiving the cartridge.

FIG. 2 is an interior view of the housing, showing an air freshener cartridge 200 containing an air freshening substance for release that is loaded into the air freshener dispenser in the cavity defined at least partially by the support member 106. The support member(s) may include any suitable supports, including arms, platforms, and other suitable structures. In one embodiment, the support member 106 includes two support arms forming a shelf for supporting a cartridge 200 (here illustrated as including cartridge body 202 and base member 208). In some embodiments, the housing further includes two lateral retaining arms 107 that further define the cavity. In certain embodiments, the support member is configured to minimally block the airflow path between the cartridge and the outlet vent, to allow for direct airflow between the cartridge and the outlet vent.

As used herein, the term “cartridge” or “air freshener cartridge” refers to a solid state substance containing an air freshening substance that is supported by a base, and which is configured to release the air freshening substance by volatilization and diffusion-based release thereof, as will be described in more detail below. For example, cartridges for use with the presently described air freshener dispensers may have any suitable composition, size, and shape to fit within the dispenser such that the desired rate and intensity of release of the air freshening substance is achieved.

The air freshener dispenser 100 also includes a fan 108 within the housing 102 that is operable to induce an airflow into and through the housing 102 and to direct the airflow to an area outside of the air freshener dispenser 100, such that released air freshening substance is entrained in the airflow directed from the air freshener dispenser 100. The active air freshener also includes a motor 109 within the housing 102 for driving the fan 108. In some embodiments, the air freshener dispenser 100 also includes a power source, such as one or more batteries, a hard wired electrical connection, solar panel(s), or other suitable power sources known in the art, to power the fan 108 and/or other components of the dispenser 100.

In certain embodiments, the dispenser does not include a control unit or similar component to direct the fan. Instead, the fan spins continuously at one given speed until the battery voltage falls to the point that it can no longer sustain the speed. As the voltage continues to fall, the fan loses speed. Eventually the voltage falls to the point where the fan no longer spins. For example, the fan may be powered by a 1.5V D-cell battery installed in the unit. In other embodiments, the dispenser may include a control unit, such as a circuit board, microchip, or other electronic or mechanical control device, so that the fan may be run intermittently and/or at various speeds. In still other embodiments, the dispenser may be hardwired to a power source outside of the dispenser.

In certain embodiments, the fan is an axial flow fan. In some embodiments, the fan is configured to run continuously when power is supplied to the motor. For example, the fan may be configured to run at a constant speed of from about 1,400 rpm to about 2,100 rpm. In other embodiments, the fan is configured to run intermittently. In certain embodiments, the fan is operable to induce an airflow rate of from about 0.1 ft³/min to about 0.5 ft³/min in the air freshener dispenser. In one embodiment, the fan is operable to induce an airflow rate of about 0.35 ft³/min in the air freshener dispenser.

The air freshener dispenser housing 102 includes the airflow outlet 104 and may also include one or more air inlet vents 111 that allow air to enter the housing 102. For example, the air inlet vents 111 may include open areas in the rear housing 102 of the dispenser 100. An airflow path from the air inlet vents 111 to the airflow outlet 104 may be defined within the housing. In certain embodiments, the airflow path includes airflow through and/or around the air freshener cartridge 200 within the cavity, so as to volatilize the air freshening substance present at one or more surfaces of the cartridge and entrain the air freshening substance in the airflow. In typical operation, airflow both around and through the channels within the cartridge will occur simultaneously.

In certain embodiments, the cavity that receives the air freshener cartridge 200 is positioned between the airflow outlet 104 and the fan 108, such that the fan 108 induces an airflow of the air that enters the dispenser through the air inlet vents 111 across and around the cartridge 200, volatilizing and entraining the air freshening substance in the airflow, and subsequently releasing it from the dispenser 100 through the airflow outlet 104. In other embodiments, the fan is positioned between the outlet and the cartridge, such that the fan pulls air through and around the cartridge. For example, the fan may be a centrifugal fan.

In certain embodiments, the housing 102 of the air freshener dispenser 100 is designed to have a particular total headspace volume. As used herein, the term “headspace” refers to the unoccupied volume of the housing. For example, the term “housing headspace” refers to the air space volume within the housing not occupied by the various components contained within the housing (e.g., fan, motor, power source, cartridge). For example, the headspace volume of the housing may be selected to accommodate a certain volume of air to attain a particular airflow release profile. In some embodiments, the housing has a headspace volume of from about 500 cm³ to about 1,500 cm³. In one embodiment, the housing has a headspace volume of about 1,000 cm³.

As discussed in more detail below, the cartridge may have airflow channels that are configured to align with the airflow path when the cartridge is loaded in the cavity, to maximize airflow through the air freshener cartridge and to increase the exposed surface area of the cartridge from which air freshening substance may be volatilized, thereby appropriately maximizing release of the air freshening substance. Thus, an air freshener dispenser 100 may be designed to provide a desired release of air freshening substance by selecting particular dispenser housing 102, as well as fan 108 parameters (e.g., volumetric air release rate), for a cartridge 200 having a particular geometry (e.g., volume, surface area) and composition (e.g., air freshening substance loading).

In certain embodiments, the cavity is designed such that a cartridge loaded in the cavity is automatically aligned with the fan and airflow outlet, to maximize release of the air freshening substance and encourage proper airflow through the cartridge and outlet. Moreover, such cavity design may provide ease of loading and/or removal of the cartridge by maintenance personnel by encouraging accurate placement of the cartridge within the housing, such that blind or low-visibility placement of the cartridge is possible (i.e., when the dispenser is mounted in a high location on a wall and it is difficult to see within the dispenser), to ensure that the cartridges are used with the appropriate dispenser (i.e., to prevent the use of cartridges designed for use with other dispensers), and to mitigate or prevent the use of unauthorized cartridge refills with the dispenser.

Air Freshener Cartridges

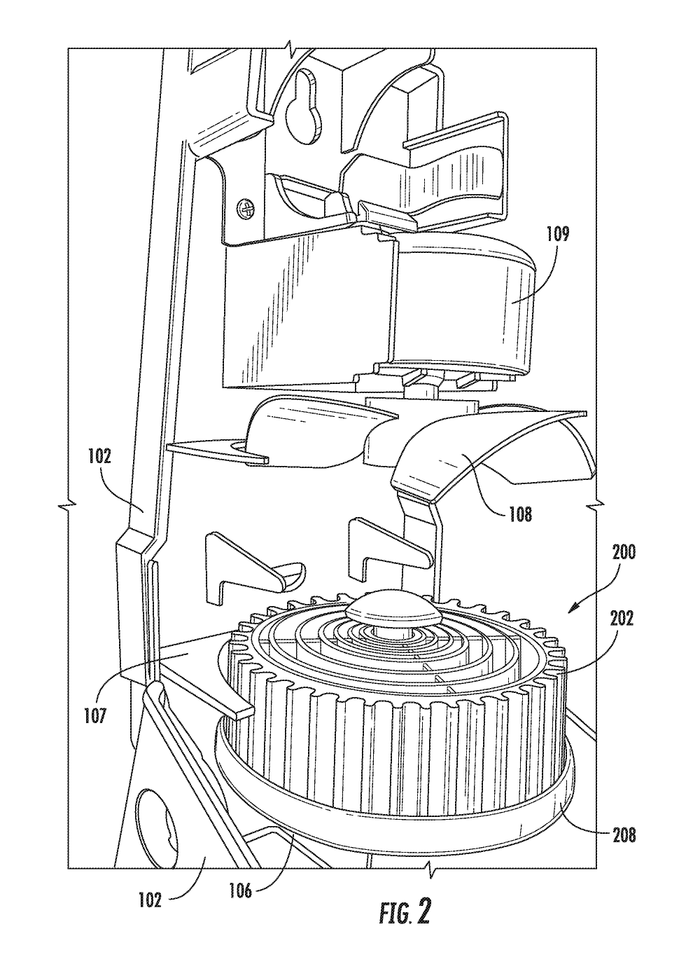

Air freshener cartridges are also provided herein. As discussed above and as shown in FIG. 3B, an air freshener cartridge 300 may include a body 302 that is any suitable solid state substance containing an air freshening substance, which is configured to release the air freshening substance by volatilization and diffusion-based release thereof, in combination with a base member 308. For example, cartridge bodies may have any suitable composition, size, and shape to fit within the dispenser such that the desired rate and intensity of release of the air freshening substance is achieved. In some embodiments, cartridge bodies for use with the presently described air freshener dispensers may include gel-based, wax-based, ceramic-based, or polymer-based substances containing or impregnated with an air freshening substance. In some embodiments, the cartridge body is a monolithic structure, such as a block or other design having airflow channels. Cartridge bodies described herein may be manufactured by any suitable means, such as by injection-molding or extrusion.

In certain embodiments, the air freshener cartridge 300 includes a body 302 containing a matrix material impregnated with an air freshening substance selected from an odor-combatting composition, a fragrance, and a combination thereof. Thus, the air freshening substance may have a scent or be unscented.

The matrix material and air freshening substance may be selected from various suitable materials known in the art. For example, the matrix material may be a polymer, such as ethylene-vinyl acetate (EVA). Suitably, EVA has no odor but can be impregnated with a fragrance or odor-combatting composition. Additionally, EVA approaches elastomeric materials in softness and flexibility, yet can be processed like a thermoplastic. In certain embodiments, the EVA polymer of the cartridge body has a number average molecular weight in the range of about 10,000 Daltons to about 100,000 Daltons, or from about 22,000 to about 87,000 Daltons. Other elastomeric or thermoplastic polymers known in the art may also be used in the cartridge body. For example, the polymer of the cartridge body may include ethyl vinyl alcohol, high density polyethylene, low density polyethylene, polystyrene, acrylic polymers, polycarbonates, polyurethanes, nylons, and mixtures and copolymers thereof.

The matrix material may be impregnated with one or more suitable air freshening substances known in the art. For example, suitable air freshening substances may be selected from those compiled by the U.S. Food and Drug Administration in Title 21 of the Code of Federal Regulations, Sections 172.5 10 and 172.5 15. In certain embodiments, the air freshening substance is selected from the group consisting of benzaldehydes, phenols, cinnamic aldehydes and esters, octadienes, dienes, cyclohexadienes, terpenes, and combinations thereof. In some embodiments, the air freshening substance includes triethylene glycol, a bleach, or hydrogen peroxide. Fragrance oils are also suitable for use alone or in combination with other fragrance chemicals. Suitable fragrance oils include, for examples spice oil, flower oil, and fruit oil. Other suitable fragrances include, but are not limited to, benzyl alcohol, ethyl maltol, furaneol, 1-hexanol, cis-3-hexen-1-ol, menthol, benzaldehyde, hexanal, cinnamaldehyde, citral, cis-3-hexenal, furfural, neral, vanillin, ethyl acetate, ethyl butanoate, ethyl decanoate, ethyl hexanoate, ethyl octanoate, hexyl acetate, isoamyl acetate, methyl butanoate, methyl salicylate, pentyl butanoate, pentyl pentanoate, sotolon, strawberry aldehyde, fructone, anethole, anisole, eugenol, dihydrojasmone, 2-acetyl-1-pyrroline, 6-acetyl-2,3,4,5-tetrahydropyridine, gamma-decalactone, gamma-nonalactone, delta-octalactone, jasmine lactone, massoia lactone, camphor, citronellol, linalool, nerol, nerolidol, alpha-terpineol, thujone, and thymol. In certain embodiments, the air freshening substance may include one or more additives, such as hindered amines or antioxidants.

In certain embodiments, the body of the cartridge contains the air freshening substance in an amount of from about 1 percent by weight to about 75 percent by weight. In some embodiments, the body contains the air freshening substance in an amount of from about 10 percent by weight to about 50 percent by weight. In some embodiments, the body contains the air freshening substance in an amount of from about 20 percent by weight to about 50 percent by weight. In some embodiments, the body contains the air freshening substance in an amount of from about 30 percent by weight to about 40 percent by weight. In one embodiment, the body contains the air freshening substance in an amount of about 25 percent by weight. In one embodiment, the body contains the air freshening substance in an amount of about 35 percent by weight. The ratio of air freshening substance to matrix material in the cartridge body may be selected to provide the desired release of the air freshening substance.

The air freshening cartridge body may be sized and shaped to provide the desired fan-induced release of the air freshener substance. Alternatively, the air freshening cartridge may be designed to passively release the fragrance, i.e., without a fan-induced airflow. For example, the cartridge body may have a particular volume and surface area to provide a desired release profile. Various cartridge body designs are illustrated at FIGS. 2, 3B, 7, and 9A-9D.

In certain embodiments, as shown in FIG. 3B, the body 302 of the cartridge 300 includes a plurality of walls 303, 304, 305, 306 defining a plurality of axial airflow channels therebetween. In some embodiments, the body includes a plurality of radial spokes and the walls are parallel walls intersecting the spokes and defining the axial airflow channels therebetween. For example, the cartridge body may include two or more radial spokes with two or more parallel walls intersecting the spokes. The parallel walls may be concentric rings, partial concentric rings, and/or other nested geometries. In some embodiments, the cartridge body is substantially cylindrical in overall shape. In some embodiments, as shown in FIG. 7, the outer concentric ring of the body 502 has a serpentine or “cog” profile, to increase the surface area of the ring.

The cartridge body may have any desired shape and geometry selected to provide to the desired release profile of the air freshening substance impregnated in the matrix material. For example, the airflow channels may extend from one edge of the body to another opposed edge of the body, such that the channels may be aligned with the airflow path within the housing when the cartridge is loaded into a cavity of a dispenser.

In certain embodiments, the cartridge body has a surface area of from about 20,000 mm² to about 55,000 mm², such as from about 45,000 mm² to 55,000 mm². In certain embodiments, the cartridge body has a volume of from about 40,000 mm³ to about 65,000 mm³. In certain embodiments, the maximum thickness of the body walls is from about 2.0 mm to about 3.5 mm.

In certain embodiments, the cartridge body is designed to have a particular surface area to volume ratio, to achieve the desired cartridge body lifespan and intensity of air freshening substance release therefrom over the lifespan. Certain parameters found to be particularly important in achieving the desired lifespan and air freshening intensity are the surface area to volume ratio and the surface area to wall thickness ratio. For example, increased air freshening substance may be released from a cartridge having increased surface area because the air freshening substance may be volatized at a higher rate. However, a cartridge having increased surface area may deplete more quickly than a cartridge of the same composition with lower surface area. Thus, the present cartridge bodies were designed for both lifespan and air freshening substance intensity, as described in more detail below and in the Example.

To keep the surface area to volume ratio unitless, and therefore transferable to standard or metric, it can be calculated by the equation:

$$\frac{\sqrt[2]{Surface Area}}{\sqrt[3]{Volume}}$$

In certain embodiments, the body has a unitless surface area to volume ratio of at least about 4. In certain embodiments, the body has a unitless surface area to volume ratio of from about 4 to about 6.25. A unitless surface area to wall thickness ratio can be calculated by the equation:

$$\frac{\sqrt[2]{Surface area}}{Wall thickness}$$

In certain embodiments, the body has a unitless surface area to wall thickness ratio of at least about 45. In certain embodiments, the body has a unitless surface area to wall thickness ratio of from about 45 to about 110, or from about 25 to about 65.

In certain embodiments, the body has a mass of from about 30 g to about 50 g, such as from about 40 g to about 45 g, or of about 42 g. In certain embodiments, the cartridge body is configured for at least 30 days of use (i.e., air freshening substance release) at acceptable levels (see Example below) when used with a continuous fan dispenser as described above. In some embodiments, the cartridge body is configured for at least 60 days of use (i.e., air freshening substance release) at acceptable levels (see Example below) when used with a continuous fan dispenser as described above. In one embodiment, the cartridge body is configured to release the air freshening substance for a period of from about 4 weeks to about 8 weeks, when exposed to continuous airflow at a volumetric rate of from about 0.1 ft³/min to about 0.5 ft³/min in the air freshener dispenser.

FIGS. 3A and 3B show exploded side and perspective views of one embodiment of an air freshener cartridge 300, having a base member 308 (also referred to as “carrier”), a body 302 (also referred to as a “refill”) and a securing mechanism 310. In certain embodiments, the base member 308 may be any structure that supports the body 302 of the cartridge 300 and provides a handling surface 311 for users, such as maintenance personnel, to load the cartridge 300 into a dispenser and unload the cartridge 300 from the dispenser.

In certain embodiments, the base member 308 includes airflow apertures that are configured to at least partially align with the airflow channels of the cartridge body 302, such that air flowing through the cartridge body 302 may also flow directly through the base member 308. In some embodiments, the base member 308 includes a support frame 309 that is in contact with a surface of the body 302 that is perpendicular to an axis of the body 302. For example, the axis of the body may be an imaginary central line of the cartridge body extending in the direction of the walls defining the axial airflow channels. That is, in embodiments in which the cartridge body includes radial spokes, the axis extends in a direction substantially perpendicular to the plane(s) in which the radial spokes extend. The support frame may define one or more airflow apertures, such that at least a portion of the axial airflow channels of the body align with the airflow apertures of the base member, so that the airflow path is oriented through the cartridge to provide optimal release of air freshening substance while minimally deflecting the airflow.

In certain embodiments, the cartridge base member 308 also includes a gripping or handling surface 311 for a user to load the cartridge into the air freshener dispenser and to unload the cartridge from the air freshener dispenser. In some embodiments, the support frame 309 includes a central portion and a plurality of radial spokes extending from the central portion to an edge. In one embodiment, the edge of the support frame forms the gripping surface for loading the cartridge into the air freshener dispenser and unloading the cartridge from the air freshener dispenser.

In certain embodiments, the base member is formed of a material that is different than the material forming the cartridge body. For example, the base member may be formed of a composition that is free of the air freshening substance contained in the body. Specifically, in embodiments in which the cartridge body is formed of a material that is difficult or unpleasant to handle (e.g., greasy, messy), the base member may beneficially be formed of a material that does not have these issues, such that maintenance personnel do not have to touch the cartridge body during loading and unloading processes. Additionally, in embodiments in which the cartridge body is formed of a material that is chemically reactive or otherwise incompatible with the material of the housing, the base member may beneficially be formed of a material that is chemically unreactive and otherwise compatible with the housing, such that the housing is not damaged due to contact with the cartridge body.

In certain embodiments, the base member is a thermoplastic or thermoset polymer. In certain embodiments, the base member is polypropylene, silicone, polycarbonate, or acrylonitrile butadiene styrene. In certain embodiments, the base member is a recyclable material. In certain embodiments, the base member has a mass of from about 4 g to about 10 g, such as about 5.7 g.

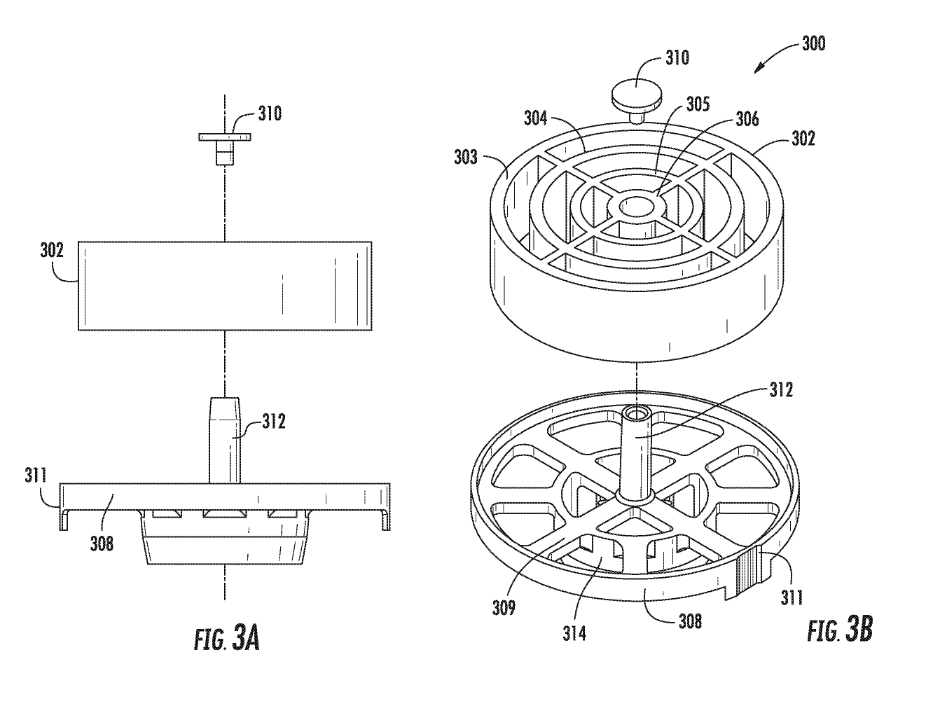

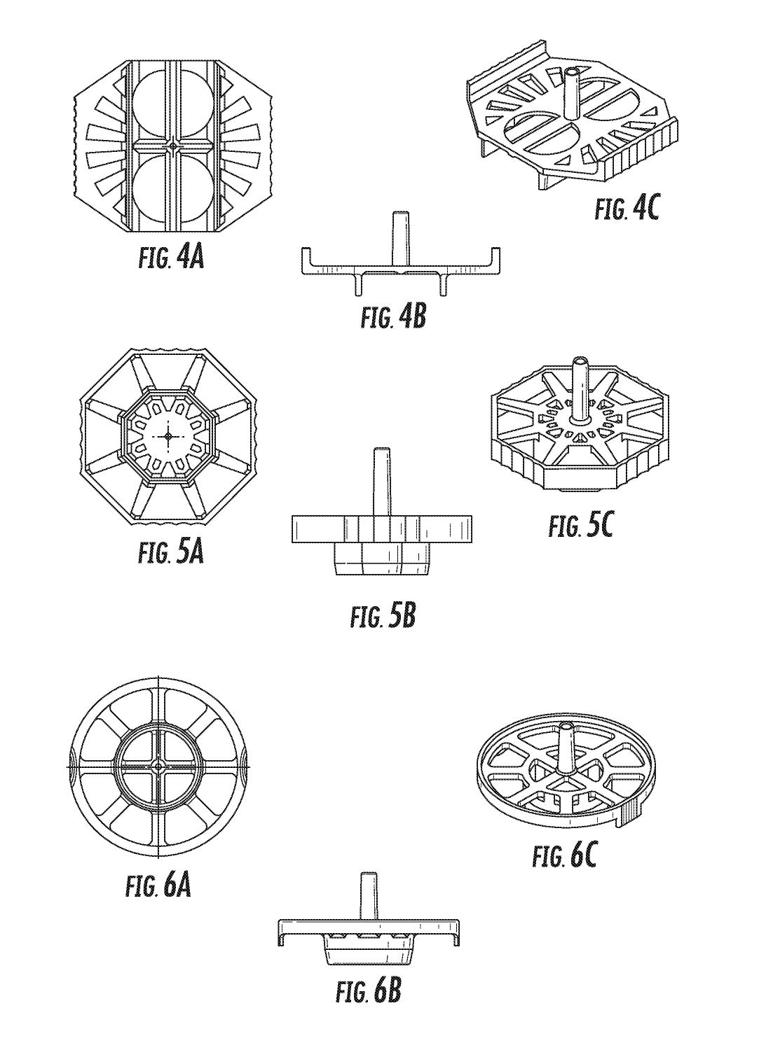

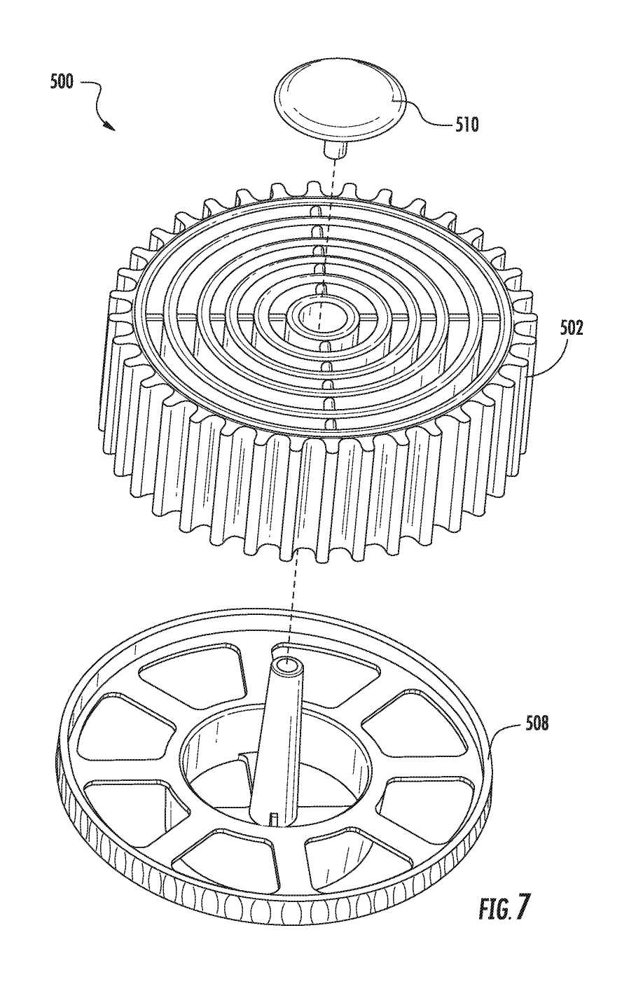

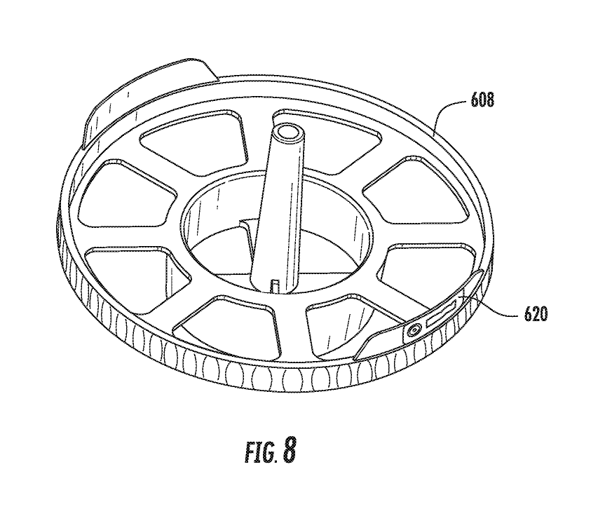

In certain embodiments, as shown in FIGS. 3A and 3B, the cartridge body 302 is removably attached to the base member 308. For example, this may facilitate use of a single base member for multiple replaceable cartridge bodies. In some embodiments, the base member 308 includes a shaft 312 extending in a direction perpendicular to the support frame 309 of the base 308 (i.e., in a direction parallel to the axis of the cartridge body) and the cartridge body 302 includes a central axial bore that receives the shaft 312 of the base member 308, to position the body 302 on the base member 308 and to limit lateral movement of the body 302 relative the base member 308. In some embodiments, the cartridge 300 also includes a securing mechanism 310 coupled to the shaft 312 of the base member 308 to secure the body 302 thereon. FIGS. 4A-4C, 5A-5C, and 6A-6C show three embodiments of base member designs for an air freshener cartridge. FIG. 7 shows an exploded perspective view of one embodiment of an air freshener cartridge 500, having a base member 508, a body 502 with an outer ring cog design, and a securing mechanism 510. In one embodiment, the securing mechanism 510 has a mass of about 0.5 g. In certain embodiments, the securing mechanism is integral with the shaft.

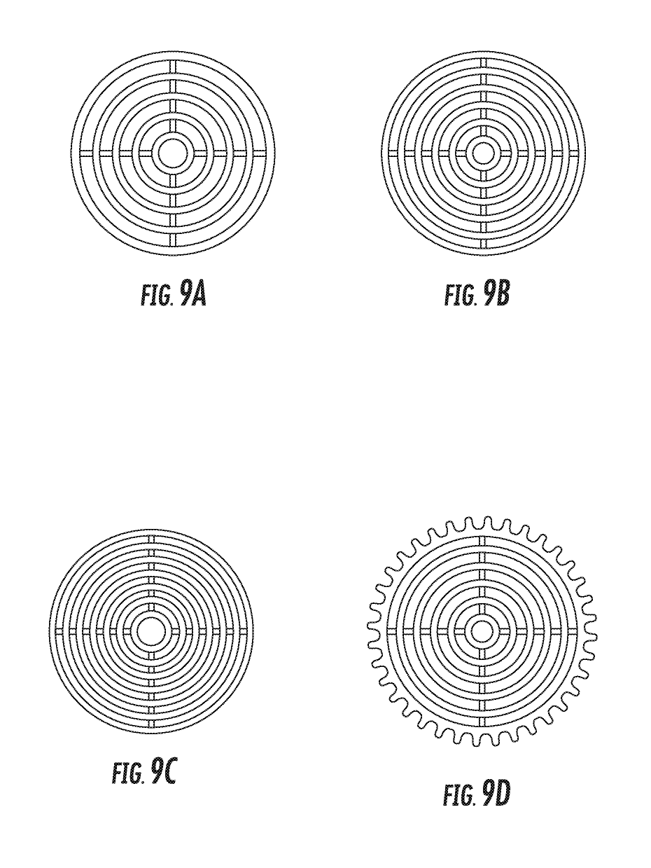

In certain embodiments, as shown in FIG. 8, the cartridge includes an end-of-life indicator 620 configured to provide a visual indication of a depletion status of the air freshening substance from the body. In some embodiments, the end-of-life indicator 620 is attached to the base member 608. In one embodiment, the end-of-life indicator is a user-activated pressure sensitive time strip. For example, the time strip may be attached or co-molded to the base member.

Assemblies

Air freshener assemblies and methods are also provided herein. In certain embodiments, an air freshener assembly includes an air freshener dispenser having any features or combination of features described herein and a cartridge having a size and shape to fit in the cavity of the dispenser to provide the desired passive and/or fan-induced release of the air freshener substance.

In certain embodiments, as shown in FIG. 2, an air freshener assembly includes an air freshener dispenser having a housing 102 with at least one support member 106 defining a cavity that removably receives a cartridge 200, a fan 108 within the housing 102 operable to induce an airflow through the aligned axial airflow channels of the body 202 and airflow apertures of the base member 208 of the cartridge 200, to direct the airflow to an area outside of the air freshener dispenser, such that released air freshening substance is entrained in the airflow directed from the air freshener dispenser, and a motor 109 within the housing for driving the fan 108. In these embodiments, the cartridge 200 may include a body 202 formed of a polymer material impregnated with an air freshening substance for release, and having a plurality of walls defining a plurality of axial airflow channels therebetween, and a base member 208 having a support frame in contact with a surface of the body 202 that is perpendicular to an axis of the body 202, the support frame defining one or more airflow apertures, such that at least a portion of the axial airflow channels of the body 202 align with the airflow apertures of the base member 208.

In certain embodiments, as shown in FIG. 2, an air freshener dispenser assembly includes an air freshener dispenser having a housing 102 with at least one support member 106 defining a cavity that removably receives the cartridge 200, such that the body 202 of the loaded cartridge does not contact the support member 106. In these embodiments, the cartridge 200 may include a body 202 formed of ethylene-vinyl acetate impregnated with an air freshening substance for release, and having a plurality of walls defining a plurality of axial airflow channels therebetween, and a base member 208 including a support frame in contact with a surface of the body 202 that is perpendicular to an axis of the body 202 and for loading the cartridge 200 into the air freshener dispenser and unloading the cartridge from the air freshener dispenser.

In certain embodiments, the assembly is configured such that the airflow through the aligned axial airflow channels of the body and airflow apertures of the base member is substantially laminar. That is, the airflow driven by the fan may be substantially unimpeded as it travels from the fan, through the cartridge body, through the cartridge base member, and out of the dispenser airflow outlet vent.

In certain embodiments, the air freshener dispenser includes a fan that is configured to run continuously and the cartridge body is designed to have a geometry and air freshening substance loading such that the desired release of air freshening substance occurs. In certain embodiments, the fan is operable to induce an airflow rate of from about 0.1 ft³/min to about 0.5 ft³/min in the air freshener dispenser. In certain embodiments, the cartridge body is designed to have a unitless surface area to volume ratio of at least about 4, such as from about 4 to about 6.25. Such dispenser and cartridge parameters may be selected so that the assembly is operable to release the air freshening substance for a period of from about 4 weeks to about 8 weeks, when exposed to continuous airflow, at an “acceptable” level of air freshening substance (see Example).

In certain embodiments, the air freshener dispenser is configured such that a loaded cartridge is positioned between the airflow outlet and the fan, such that the air freshening substance, which may be incompatible with the material forming the fan, does not come into contact with the fan (i.e., airflow with entrained air freshening substance is directed away from the fan).

In certain embodiments, the cartridge base member material is selected such that it is unreactive with the material of the air freshener dispenser housing material, especially the material of the support member(s). In one embodiment, the base member is formed of a material that is chemically unreactive with a material forming the support member. In one embodiment, the base member and the support member are formed of the same material. In some embodiments, the support member 106 includes two support arms forming a shelf for supporting a cartridge. In certain embodiments, the housing 102 also includes two lateral retaining arms 107 that further define the cavity. In some embodiments, the lateral retaining arms and cartridge body are designed such that the body of a cartridge loaded in the cavity does not contact the lateral retaining arms. In some embodiments, the housing and cartridge are configured such that the body of a cartridge loaded in the cavity does not contact any portion of the housing.

In certain embodiments, as shown in FIG. 3B, the cartridge base member 308 include a projection 314 extending from a surface of the support frame 308 opposite the cartridge body 302. In certain embodiments, the projection 314 corresponds to a slot formed by the two support arms 106 (see FIG. 1C), such that the projection 312 and slot guide loading of the cartridge into the cavity. Such feature may assist with blind or low-visibility loading of the cartridge into the dispenser, such as for high wall-mounted dispensers.

Thus, parameters of the assembly, including, but not limited to, the cartridge body composition (e.g., air freshening substance to matrix material ratio), the cartridge body size and shape (e.g., volume and surface area), the housing design, and the volumetric air release rate and fan speed, may be selected to achieve a particular air freshener release profile for the assembly. For example, these parameters may be tailored to achieve release of a consistent amount of air freshening substance to combat odor and/or provide fragrance over a desired lifetime of the cartridge, without depleting the cartridge too quickly or providing overwhelming or unpleasant fragrance release.

In certain embodiments, the assembly parameters are configured such that the lifetime of the cartridge in the air freshener dispenser at regular usage rates is from 7 days to 60 days. For example, the assembly may be configured such that the lifetime of the cartridge in the air freshener dispenser at regular usage rates is about 7 days, about 14 days, about 30 days, or about 60 days, depending on the desired maintenance schedule and other considerations.

In certain embodiments, the assembly is configured to provide comparable or improved air freshening substance dispense as compared to existing continuous fan dispensers. Such dispensers typically use a liquid or gel cup style cartridge, such that the fan blows air only across the surface of the gel or liquid provided in the cup, resulting in turbulent and unpredictable airflow from the dispenser. The present disclosure advantageously provides a cartridge design that allows air to travel through the cartridge, limiting the turbulent airflow and increasing the surface area contacting the airflow, to provide controlled, predictable airflow from the dispenser, which increases the effective range of the assembly. Because only the air freshening substance at the surface of the cartridge body can volatize, increasing the surface area of the body improves release of the air freshening substance, as compared with limited surface area embodiments, such as a conventional gel cup. With limited diffusion in thick molded designs, only a portion of the fragrance is utilized upon replacement and disposal. This results in a shortened life and more frequent replacement schedule for end users or the need to use more material to maintain life.

Thus, the present cartridge bodies advantageously address the limitation of unused air freshening substance embedded within the body by maximizing the surface area of the body while minimizing the overall volume or, more specifically, the wall thickness. Evaporation is a surface phenomenon, so maximizing the surface area to volume or wall thickness ratio facilitates more complete utilization of the air freshening substance. Near complete utilization provides constant release over a life—a life that is maintained or extended compared to a configuration with lower surface area to volume or thickness ratio. The cartridge body geometries described herein and in the below Example provide a body lifespan similar or greater than other continuous fan dispenser cartridge designs, while also providing a suitable air freshening substance release over time.

Kits are also provided herein, including an air freshener cartridge as described in any embodiments or combination of embodiments herein and an air freshener dispenser as described in any embodiments or combination of embodiments herein.

Methods for reducing malodor in a public washroom are also provided. These methods may include, for instance, (i) providing an air freshener dispenser as described in any embodiments, or combination of embodiments, herein, (ii) loading the dispenser with an air freshener cartridge as described in any embodiments, or combination of embodiments, herein, and (iii) directing airflow containing released air freshening substance from the air freshener dispenser.

### EXAMPLES

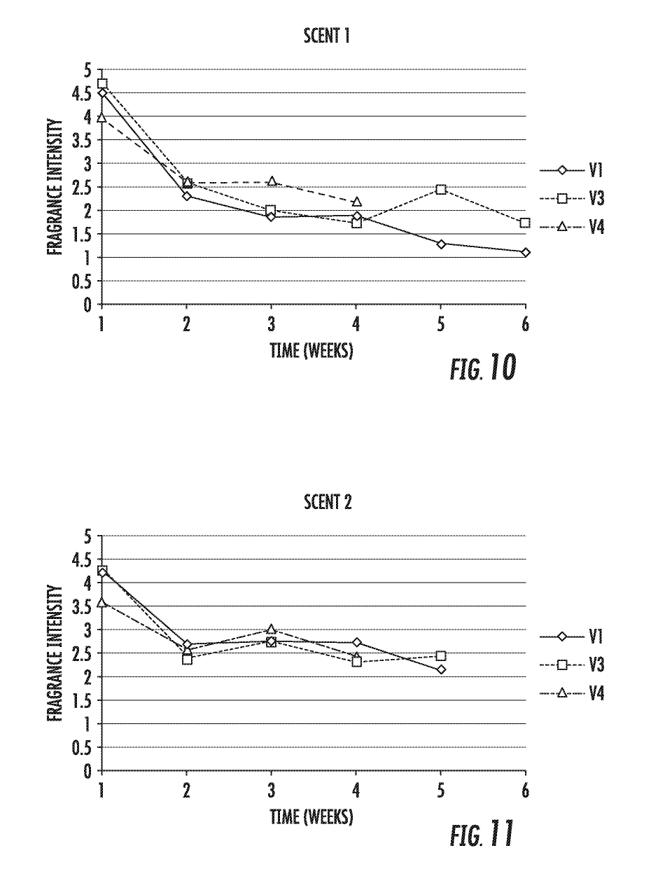

Air freshener cartridges and dispensers having a variety of designs and parameters were manufactured and tested for performance characteristics, including fragrance intensity and air freshener longevity. FIGS. 9A-9D show four embodiments (V1-V4) of cartridge body portions of air freshener cartridges that were manufactured and tested. The geometrical parameters of cartridge bodies V1-V4 are shown in Table 1. Cartridge bodies V1-V3 are similar except for the number of rings, and therefore the surface area. V4 includes the “cog” design around the outside to further increase surface area and increased the mass and the fragrance load.

TABLE 1
Geometrical Parameters of Cartridge Bodies V1-V4.

| | | | | | Wall | Wall | | | |
|---|---|---|---|---|---|---|---|---|---|
| | | | Surface | | thickness | thickness | | Max | Min |
| Cartridge | Fragrance | Mass | area | Volume | min | max | Unitless | Unitless | Unitless |
| Body | load (%) | (g) | (mm²) | (mm³) | (mm) | (mm) | Sa:V | SA:WT | SA:WT |
| V1 | 25 | 32 | 24655 | 61060 | 2.47 | 3.5 | 4.0 | 63.6 | 44.9 |
| (FIG. 9A) | | | | | | | | | |
| V2 | 25 | 32 | 28575 | 61060 | 1.98 | 3.0 | 4.3 | 79.3 | 52.3 |
| (FIG. 9B) | | | | | | | | | |
| V3 | 25 | 32 | 32566 | 61060 | 1.69 | 2.6 | 4.6 | 92.9 | 60.4 |
| (FIG. 9C) | | | | | | | | | |
| V4 | 35 | 42 | 48562 | 44281 | 2.03 | 3.0 | 6.2 | 77.3 | 52.3 |
| (FIG. 9D) | | | | | | | | | |

The cartridges were loaded into dispensers as shown in FIGS. 1A-1D and 2, with a continuous fan, and the dispensers were placed in separate evaluation booths of 288 cubic feet. The evaluation booths were maintained at a temperature of between 68-72° F. The humidity within the booths was monitored to ensure consistency (typically between 20-40% RH). Eight trained panelists evaluated the fragrance intensity within the booth once per week over eight weeks, and the results were averaged to determine the average intensity over time. The fragrance intensity was evaluated on a 5 point scale with “5” indicating the greatest fragrance intensity and a near overwhelming fragrance level, and “0” indicating no fragrance detected. Intensity values of 2-4 were considered acceptable fragrance levels. Intensity values of less than 2 were considered minimally effective.

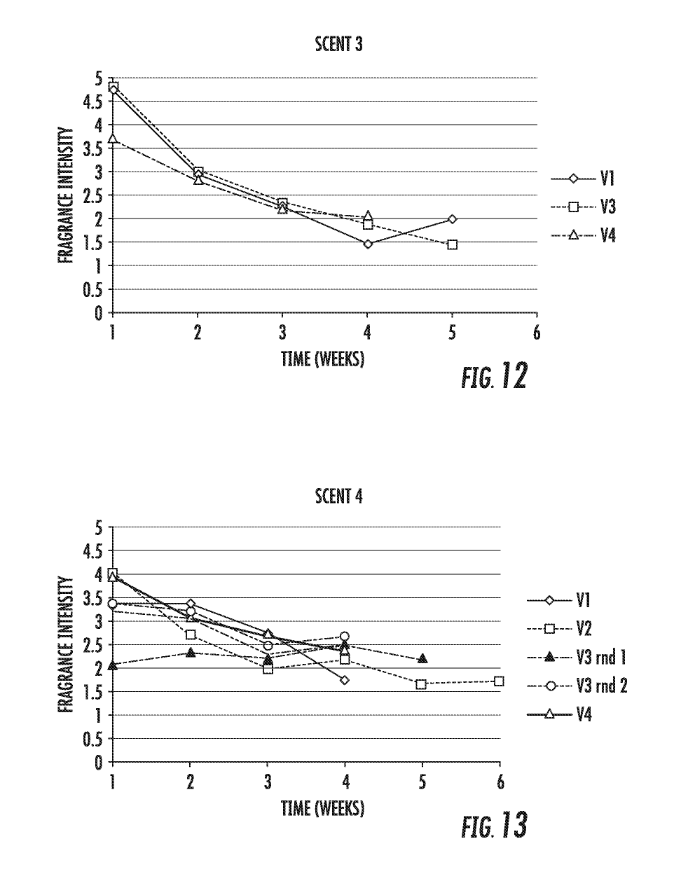

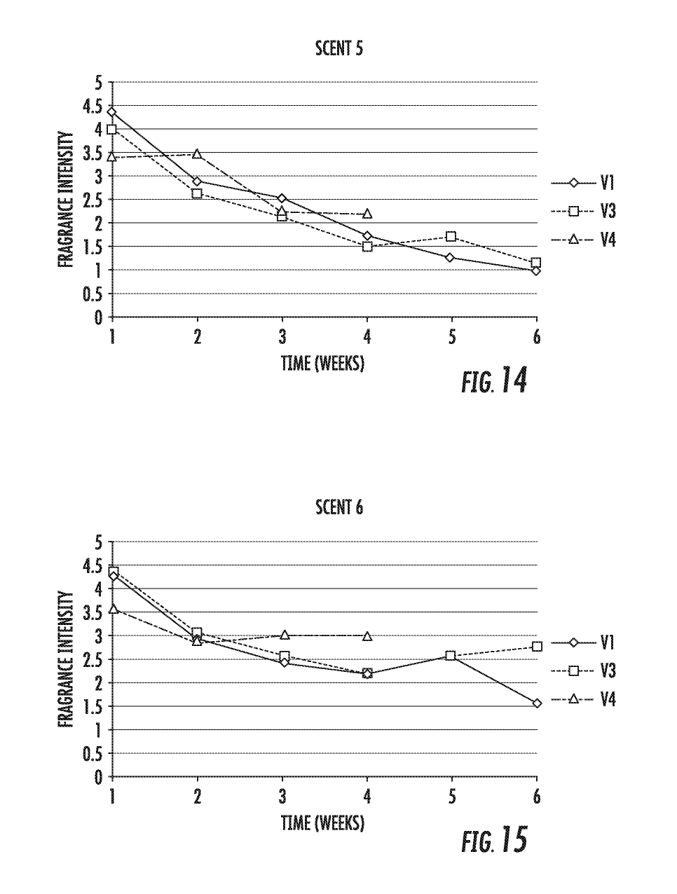

Various fragrances were used in the cartridge, as shown in Table 2, which includes the average intensity levels as evaluated during the tests. The fragrances considered relatively strong includes Scents 1 and 2, while the fragrances considered moderate include Scents 3 and 4, and the fragrances considered relatively light include Scents 5 and 6. The test results are also represented in the graphs of FIGS. 10-15, which show the measured fragrance intensity over time.

Generally, the test results show that for cartridge embodiments having the same volume, mass, and fragrance loading, but having different surface area to volume ratios, the cartridges with the higher surface area show a slightly higher intensity later in the lifetime of the cartridge.

Across the various fragrances, the cartridges having the highest unitless surface area to volume ratio (i.e., V4) maintain a higher fragrance intensity (in the “acceptable fragrance level” range) for a longer time period. That is, the fragrance intensity curve decreases at a slower rate than others (slope is less negative). Thus, the lifetime of cartridges having this surface area to volume ratio is extended past the lifetime of cartridge having lower ratios. Surprisingly, despite the use of a continuous fan, the lifetime of cartridges having surface to volume ratios of 4 or greater, was found to be at least 4 weeks at acceptable fragrance levels. Thus, these cartridges may be replaced at a similar maintenance interval to conventional gel cup style cartridges for continuous dispensers, and provide enhanced fragrance levels. These performance curves were unexpected for cartridges having a lifespan in the range of conventional gel cups. In particular, conventional gel cups may have a lifespan in the range of 60 days. Cartridges V1-V3 displayed fragrance intensity at similar or slightly lower levels, as compared to conventional gel cups, over the course of 8 weeks, while cartridge V4 generally displayed higher fragrance intensity and a slower decrease in fragrance intensity, as compared to cartridges V1-V3.

Additionally, the increased surface area to volume provides a lower initial fragrance intensity, whereas the lower surface area to volume cartridges showed an initial fragrance intensity in or near the overpowering range.

TABLE 2
Intensity Test Results for Various Fragrance Cartridges.

| Cartridge | | | | | | | | |
|---|---|---|---|---|---|---|---|---|
| Body | | Mass | | Week | Week | Week | Week | Week |
| Version | Fragrance | (g) | Initial | 1 | 2 | 4 | 6 | 8 |
| V1 | Scent 4 | 32 | 3.38 | 3.38 | 2.75 | 1.78 | | |
| V2 | Scent 4 | 32 | 4 | 2.71 | 2 | 2.25 | 1.71 | 1.75 |
| V3 | Scent 4 | 32 | 3.83 | 2.14 | 2.38 | 2.25 | 2.57 | 2.25 |
| V3 | Scent 4 | 32 | 3.38 | 3.25 | 2.5 | 2.67 | | |
| V4 | Scent 4 | 42 | 4 | 3.13 | 2.75 | 2.44 | | |
| V1 | Scent 1 | 32 | 4.5 | 2.29 | 1.88 | 1.88 | 1.29 | 1.13 |
| V3 | Scent 1 | 32 | 4.67 | 2.57 | 2 | 1.75 | 2.43 | 1.75 |
| V4 | Scent 1 | 42 | 4 | 2.63 | 2.63 | 2.22 | | |
| V1 | Scent 3 | 32 | 4.75 | 2.88 | 2.29 | 1.43 | 2 | |
| V3 | Scent 3 | 32 | 4.75 | 3 | 2.29 | 1.86 | 1.43 | |
| V4 | Scent 3 | 42 | 3.71 | 2.85 | 2.25 | 2.11 | | |
| V1 | Scent 2 | 32 | 4.25 | 2.63 | 2.71 | 2.71 | 2.14 | |
| V3 | Scent 2 | 32 | 4.25 | 2.38 | 2.71 | 2.29 | 2.43 | |
| V4 | Scent 2 | 42 | 3.57 | 2.57 | 3 | 2.44 | | |
| V1 | Scent 5 | 32 | 4.37 | 2.88 | 2.5 | 1.75 | 1.28 | 1 |
| V3 | Scent 5 | 32 | 4 | 2.63 | 2.13 | 1.5 | 1.71 | 1.14 |
| V4 | Scent 5 | 42 | 3.43 | 3.5 | 2.29 | 2.22 | | |
| V1 | Scent 6 | 32 | 4.25 | 2.88 | 2.38 | 2.19 | 2.57 | 1.57 |
| V3 | Scent 6 | 32 | 4.38 | 3 | 2.5 | 2.19 | 2.57 | 2.75 |
| V4 | Scent 6 | 42 | 3.57 | 2.88 | 3 | 3 | | |

The test results demonstrate that the geometry, mass, and fragrance loading of the cartridge body has a significant impact on lifespan and fragrance intensity, and that these parameters may be tailored to achieve consistent release of a pleasant level of fragrance. For example, cartridges having a unitless surface area to volume ratio of about 4 or more, or more particularly of about 6 or more, with a fragrance loading of about 20-40 percent, display lifespans of at least 4 weeks at acceptable fragrance levels, when exposed to a continuous fan.

Accordingly, air freshener dispensers, cartridges, and assemblies may be designed to achieve desired air freshener release in settings such as washrooms, washroom stalls, and other locations where malodor is a common issue. The air freshener dispensers, cartridges, and assemblies described herein may have one or more of the following benefits over currently available options: (1) improved effectiveness because the dispenser/freshener releases the air freshener in a controlled and predictable manner; (2) easier refillability because the dispenser and cartridge may include a self-locating feature; (3) easier cartridge handling and refillability because the cartridge includes a base for handling the air freshener; (4) easier maintenance because the dispenser/freshener does not require a liquid refill; and (5) improved cartridge lifespan and fragrance intensity over the lifespan.

While the disclosure has been described with reference to a number of example embodiments, it will be understood by those skilled in the art that the invention is not limited to such embodiments. Rather, the invention can be modified to incorporate any number of variations, alterations, substitutions, or equivalent arrangements not described herein, but which are commensurate with the spirit and scope of the invention. Additionally, while various example embodiments of the invention have been described, it is to be understood that aspects of the invention may include only some of the described embodiments. Accordingly, the invention is not to be seen as limited by the foregoing description, but is only limited by the scope of the appended claims.

We claim:

1. An air freshener cartridge for use in an air freshener dispenser, comprising:
a body formed of a polymer material impregnated with an air freshening substance for release, the body comprising a plurality of radial spokes and a plurality of parallel walls intersecting the spokes and defining a plurality of axial airflow channels therebetween; and
a base member comprising a support frame in contact with a surface of the body that is perpendicular to an axis of the body, the support frame defining one or more airflow apertures, such that at least a portion of the axial airflow channels of the body align with the airflow apertures of the base member,
wherein the base member comprises a gripping surface for a user to load the cartridge into the air freshener dispenser and to unload the cartridge from the air freshener dispenser.

2. The cartridge of claim 1, wherein the parallel walls comprise concentric rings.

3. The cartridge of claim 1, wherein:
the support frame comprises a central portion and a plurality of radial spokes extending from the central portion to an edge, the spokes defining the one or more airflow apertures, and
the edge forms the gripping surface.

4. The cartridge of claim 1, wherein the polymer material comprises ethylene-vinyl acetate.

5. The cartridge of claim 1, wherein the body comprises the air freshening substance in an amount of from about 20 percent by weight to about 50 percent by weight.

6. The cartridge of claim 1, wherein the body has a unitless surface area to volume ratio of from about 4 to about 6.25.

7. The cartridge of claim 1, wherein a maximum thickness of the parallel walls is from about 2.0 mm to about 3.5 mm.

8. The cartridge of claim 1, wherein the body is configured to release the air freshening substance for a period of from about 4 weeks to about 8 weeks, when exposed to continuous airflow at a volumetric rate of from about 0.1 ft³/min to about 0.5 ft³/min in the air freshener dispenser.

9. The cartridge of claim 1, wherein the body is removably attached to the base member.

10. The cartridge of claim 1, wherein:
the base member further comprises a shaft extending in a direction perpendicular to the support frame of the base member, and
the body further comprises a central axial bore that receives the shaft of the base member, to position the body on the base member and to limit lateral movement of the body relative the base member.

11. The cartridge of claim 10, wherein the cartridge further comprises a securing mechanism coupled to the shaft of the base member to secure the body thereon.

12. The cartridge of claim 1, wherein the base member comprises polypropylene, polycarbonate, or acrylonitrile butadiene styrene.

13. An air freshener cartridge for use in an air freshener dispenser, comprising:
a body formed of ethylene-vinyl acetate impregnated with an air freshening substance for release, the body comprising a plurality of walls defining a plurality of axial airflow channels therebetween; and
a base member comprising a support frame in contact with a surface of the body and for loading the cartridge into the air freshener dispenser and unloading the cartridge from the air freshener dispenser, the base member formed of a composition free of the air freshening substance,
wherein the body has a unitless surface area to volume ratio of at least about 4.

14. An air freshener dispenser assembly, comprising:
an air freshener cartridge that comprises:
a body formed of a polymer material impregnated with an air freshening substance for release, the body comprising a plurality of walls defining a plurality of axial airflow channels therebetween; and
a base member comprising a support frame in contact with a surface of the body that is perpendicular to an axis of the body, the support frame defining one or more airflow apertures, such that at least a portion of the axial airflow channels of the body align with the airflow apertures of the base member; and
an air freshener dispenser that comprises:
a housing comprising at least one support member defining a cavity for removably receiving the cartridge;
a fan within the housing operable to induce an airflow through the aligned axial airflow channels of the body and airflow apertures of the base member, to direct the airflow to an area outside of the air freshener dispenser, such that released air freshening substance is entrained in the airflow directed from the air freshener dispenser; and
a motor within the housing for driving the fan.

15. The assembly of claim 14, wherein the fan comprises an axial-flow fan.

16. The assembly of claim 14, wherein the fan is configured to run continuously.

17. The assembly of claim 16, wherein the fan is configured to run at a constant speed of from about 1,400 rpm to about 2,100 rpm.

18. The assembly of claim 14, wherein the fan is configured to run intermittently.

19. The assembly of claim 14, wherein the housing has a headspace volume of from about 500 cm³ to about 1,500 cm³.

20. The assembly of claim 14, wherein the housing further comprises an airflow outlet through which the airflow comprising the entrained released air freshening substance is directed from the air freshener dispenser.

21. The assembly of claim 20, wherein the cavity that receives the cartridge is positioned between the airflow outlet and the fan.

22. The assembly of claim 20, wherein the airflow outlet comprises an open area of from about 1,000 mm² to about 1,500 mm².

23. The assembly of claim 14, wherein the fan is configured to induce an airflow rate of from about 0.1 ft³/min to about 0.5 ft³/min in the air freshener dispenser.

24. The assembly of claim 23, wherein the assembly is configured such that the body releases the air freshening substance for a period of from about 4 weeks to about 8 weeks, when exposed to continuous airflow.

25. The assembly of claim 14, wherein the assembly is configured such that the airflow through the aligned axial airflow channels of the body and airflow apertures of the base member is substantially laminar.

26. An air freshener dispenser assembly, comprising:
an air freshener cartridge that comprises:
a body formed of ethylene-vinyl acetate impregnated with an air freshening substance for release, the body comprising a plurality of walls defining a plurality of axial airflow channels therebetween; and
a base member comprising a support frame in contact with a surface of the body that is perpendicular to an axis of the body and for loading the cartridge into the air freshener dispenser and unloading the cartridge from the air freshener dispenser; and
an air freshener dispenser that comprises:
a housing comprising at least one support member defining a cavity for removably receiving the cartridge, such that the body of a cartridge loaded in the cavity does not contact the support member;
a fan within the housing operable to induce an airflow through the axial airflow channels of the body, and to direct the airflow to an area outside of the air freshener dispenser, such that released air freshening substance is entrained in the airflow directed from the air freshener dispenser; and
a motor within the housing for driving the fan.
